(19) United States
(12) Patent Application Publication
(10) Pub. No.: US 2011/0224443 A1
(43) Pub. Date: Sep. 15, 2011

(54) PREPARATION OF FOSAMPRENAVIR CALCIUM

(76) Inventors: Venkata Naga Brahmeshwara Rao Mandava, Hyderabad (IN); Praveen Cherukupally, Hyderabad (IN); Ganesh Varanasi, Hyderabad (IN); Srinivas Areveli, Karimnagar (IN); Chandra Sekhar Vempati, Vijayawada (IN); Ramakrishna Reddy Narayana, Hyderabad (IN); Sreenadha Charyulu Kandala, Hyderabad (IN)

(21) Appl. No.: 13/047,552

(22) Filed: Mar. 14, 2011

Related U.S. Application Data
(60) Provisional application No. 61/348,012, filed on May 25, 2010.

(30) Foreign Application Priority Data
Mar. 15, 2010 (IN) 692/CHE/2010

(51) Int. Cl. C07F 9/6561; C07D 209/44; C07F 9/655

(52) U.S. Cl. 548/415; 548/470; 549/222

(57) ABSTRACT

Process for preparation of fosamprenavir and its intermediate salts using novel phthalimide intermediates.

The invention relates to processes for preparing fosamprenavir (I) and its pharmaceutically acceptable salts. Other aspects of the present invention relate to intermediate compounds and their use in the preparation of fosamprenavir.

Fosamprenavir calcium has a chemical name (3S)-tetrahydrofuran-3-yl-(1S,2R)-3-[[(4-aminophenyl)sulfonyl] (isobutyl)amino]-1-benzyl-2-(phosphonooxy) propyl carbamate monocalcium salt, and is structurally represented by Formula I.

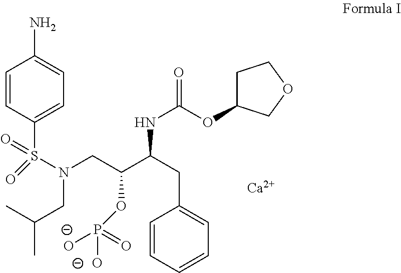

The anti-viral compound fosamprenavir calcium is a HIV protease inhibitor. It is a prodrug of an antiviral compound amprenavir and is indicated for the treatment of HIV-1 infection in combination with other antiretroviral agents.

U.S. Pat. No. 6,436,989 B1 discloses fosamprenavir calcium and other salts of fosamprenavir such as sodium, potassium and magnesium, their pharmaceutical compositions and methods of treating HIV infection and inhibiting aspartyl protease activity in a mammal. U.S. Pat. No. 6,514,953 B1 discloses polymorphic Form I of fosamprenavir calcium, its pharmaceutical composition, and its method of use for treatment of an HIV infection. The patent also discloses a process for the preparation of fosamprenavir and its salts involving reaction of a compound of Formula A with a phosphorylating agent to give the intermediate of Formula B, followed by reduction of the obtained intermediate of Formula B to give fosamprenavir. Fosamprenavir is converted to its calcium salt by dissolving fosamprenavir in a suitable solvent and adding a solution of water and a source of calcium ions to it. The process can be represented schematically as shown below in Scheme-A.

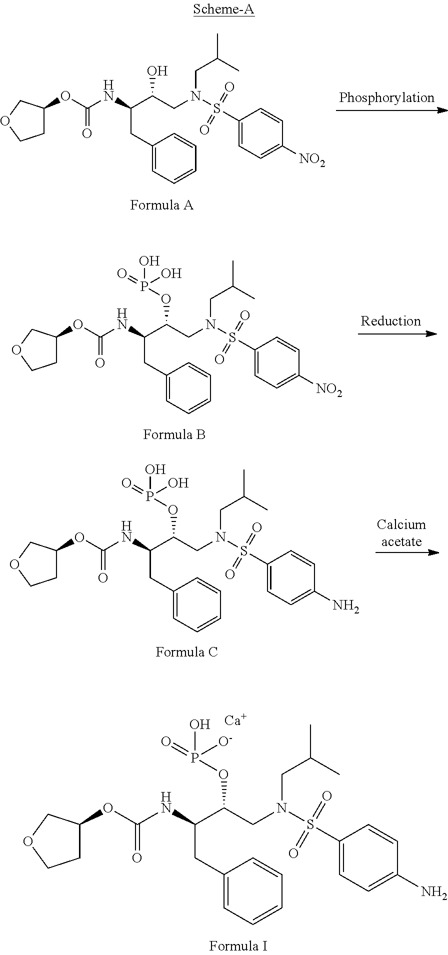

WO2010134045 A1 discloses amorphous form of fosamprenavir calcium and a process for its preparation and also states that the amorphous form has appreciable solubility. WO2011001383 A1 discloses crystalline Form II of fosamprenavir calcium and a process for its preparation.

Given the increasing therapeutic value of fosamprenavir and its salts, new synthetic routes are desirable to prepare fosamprenavir, and its polymorphic forms. The present application provides an alternate process for the preparation of fosamprenavir and its salts which is cost effective, safe and can be practiced on an industrial scale.

### SUMMARY

In one aspect, the invention provides methods for preparing fosamprenavir (I) and its pharmaceutically acceptable salts. In particular aspects, the present invention relates to intermediate compounds and their use in the preparation of fosamprenavir and its pharmaceutically acceptable salts.

### BRIEF DESCRIPTION OF THE DRAWING

FIG. 1 is an illustrative X-ray powder diffraction pattern of substantially amorphous fosamprenavir calcium.

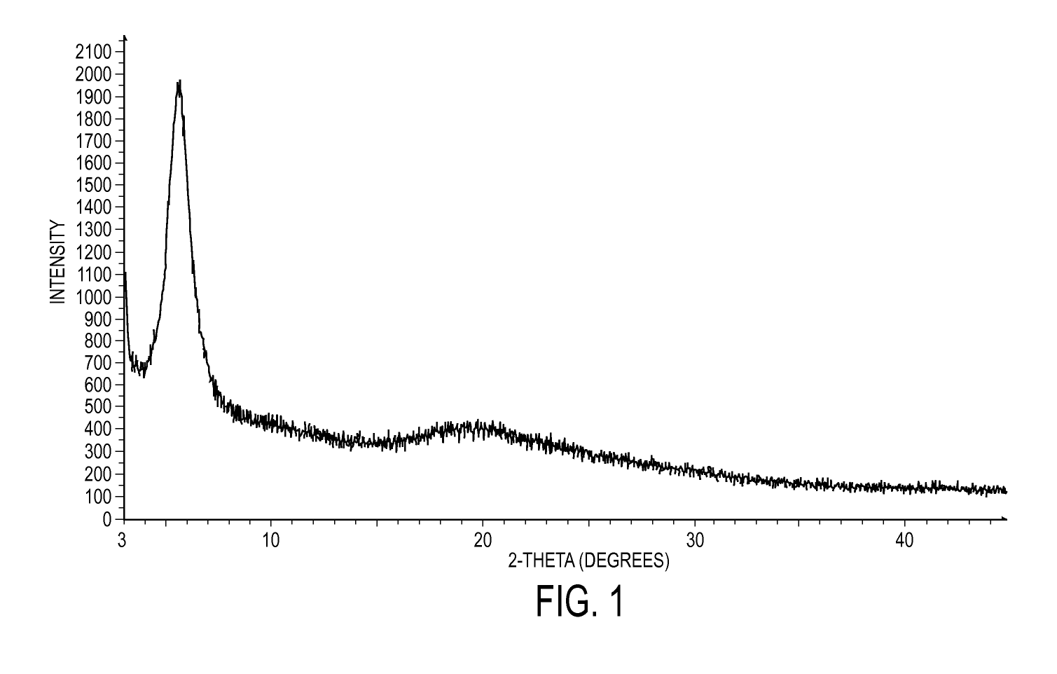

FIG. 2 is an illustrative Differential Scanning calorimetry of substantially amorphous fosamprenavir calcium.

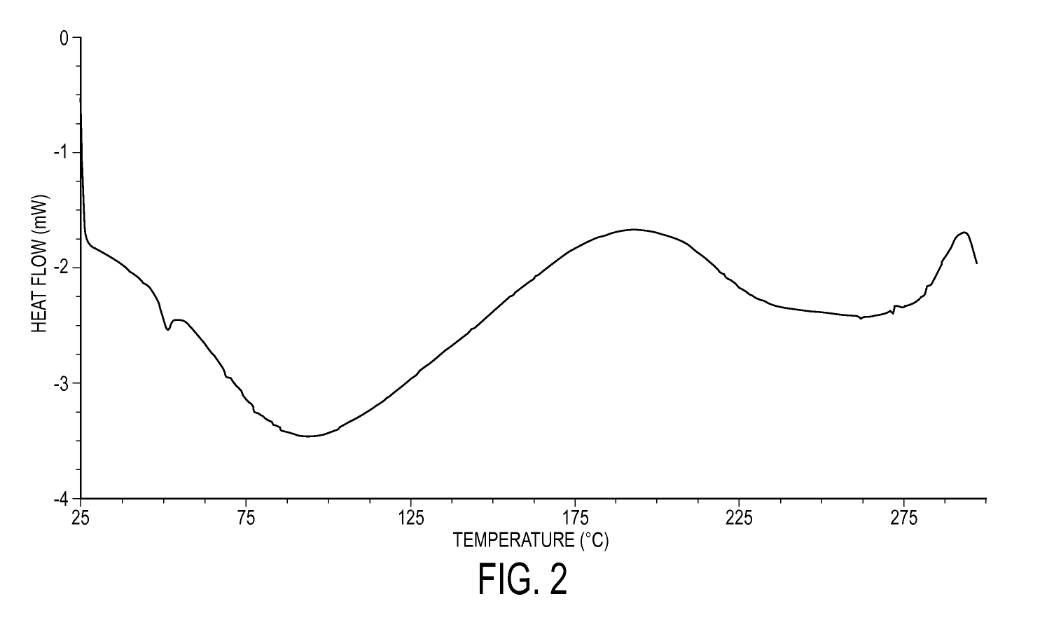

### DETAILED DESCRIPTION

In one aspect, the present application provides a process for the preparation of amine-protected N-((2R,3S)-3-amino-2-hydroxy-4-phenylbutyl)-4-(1,3-dioxoisoindolin-2-yl)-N-isobutylbenzenesulphonamide of Formula II comprising reacting an amino alcohol of Formula III with 4-phthalimidobenzene sulfonyl halide of Formula IV.

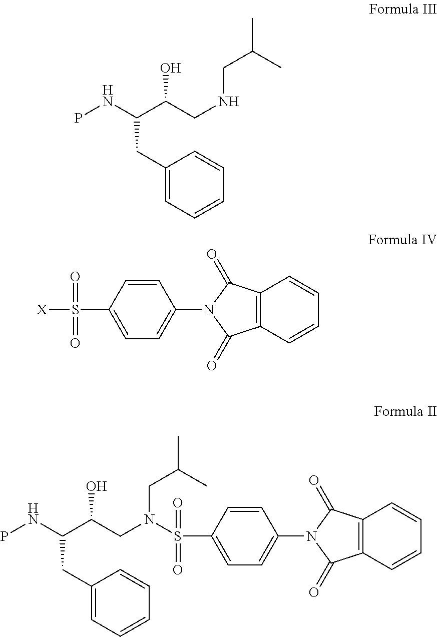

wherein X is any halogen, e.g., Cl, Br, or I, and P is any amine protecting group.

Suitable solvents which can be used for the reaction include, but are not limited to ketone solvents such as acetone, ester solvents such as ethyl acetate, ether solvents such as diethyl ether, an amide solvent such as dimethylformamide or dimethylacetamide, halogenated hydrocarbon solvents, such as dichloromethane, other solvents such as acetonitrile or toluene, or any mixtures thereof. The 4-phthalimidebenzenesulphonyl halide of Formula IV may be any halide such as 4-phthalimidebenzenesulphonyl chloride or 4-phthalimidebenzenesulphonyl bromide. Suitable temperatures for conducting the reaction range from about 20° C. to about 100° C. or from about 50° C. to about 70° C.

The compound of Formula II can be isolated from the reaction mixture by techniques well known in the art. The compound is suitably isolated by evaporating off the reaction mixture and adding water to precipitate the product. The product can be isolated by filtration. The isolated compound may be further purified by precipitation from a suitable solvent or a mixture of solvents. The compound of Formula II can be characterized by its mass spectral m/z values. The compound can be isolated as a crystalline solid and can be used for preparation of fosamprenavir and its salts.

In another aspect, the present application provides a process for the preparation of fosamprenavir starting from the intermediate of Formula II comprising:
a) deprotecting the protected compound of Formula II in an acidic media to yield a deprotected amino derivative of Formula V or a salt thereof:

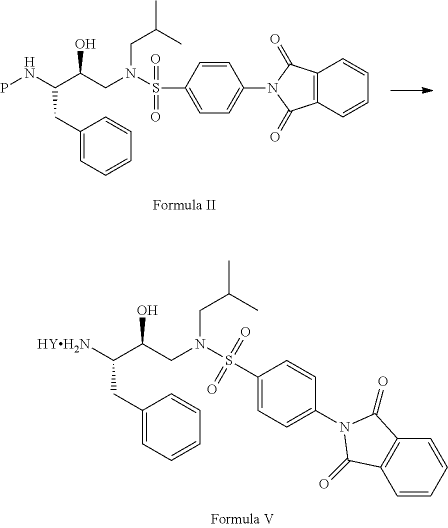

wherein Y is any acid addition salt-forming residue;
b) reacting the amino derivative of Formula V with (S)-tetrahydro-3-furanol and a carbonylation agent, to produce the phthalimide derivative of Formula VI;

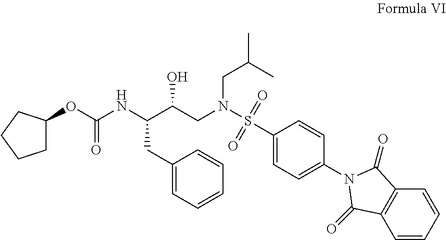

c) reacting the derivative of Formula VI with a phosphorylating agent to obtain a phthalimide-protected fosamprenavir of Formula VII;

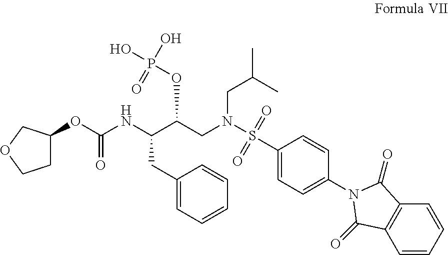

d) deprotecting the compound of Formula VII, to obtain fosamprenavir of Formula VIII; and

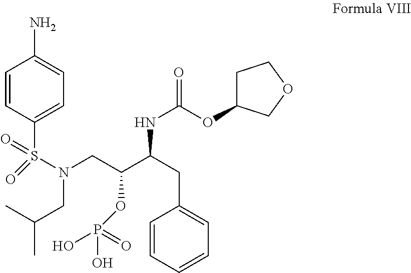

e) optionally, converting fosamprenavir to its pharmaceutically acceptable salt.

Suitable protecting groups represented as P in Formula II include, but are not limited to amine protecting groups such as alkyl, aryl, benzyl or heteroaryl carbamates, alkyl or aryl amides, silyl groups, tosylate, and mesylate. Suitable acids which may be used for providing the acidic media may be selected from inorganic acids such as hydrochloric acid, gaseous hydrogen chloride, trifluoroacetic acid, triflic acid, methanesulfonic acid, or the like.

Derivatives of (S)-tetrahydro-3-furanol which may be used in step b) are made by reaction with carbonylation agents. Suitable derivatives of tetrahydrofuranol which may be used in step b) include tetrahydrofuryloxycarbonyls, prepared for example, by reacting (S)-tetrahydro-3-furanol with 1,1′-carbonyldiimidazole, a chloroformate, or triphosgene. Suitably, this step may also be carried out by reacting the product of step a) with phosgene or an equivalent, to produce an isocyanate intermediate, which can then react with (S)-tetrahydro-3-furanol or a precursor. Suitable phosphorylating agents which can be used in step c) include, but are not limited to, phosphorus oxychloride, phosphorus pentachloride, and dibenzylchlorophosphate. The reaction is suitably conducted in the presence of a base such as pyridine, triethylamine, diisopropylethylamine, or the like, and optionally in the presence of a solvent. Suitable deprotecting agents which can be used in step d) include bases such as methylamine, n-butylamine, isobutylamine, isopropylamine, hydrazine hydrate, sodium hydroxide, or the like. Suitable solvents which can be used for conducting reactions of steps a) to e) include, but are not limited to halogenated hydrocarbon solvents such as dichloromethane; hydrocarbon solvents such as toluene, xylene, ortho-xylene, n-heptane, n-hexane, cyclohexane, methylcyclohexane, or the like; ester solvents such as ethyl acetate, n-propyl acetate, n-butyl acetate, t-butyl acetate, or the like; aprotic polar solvents such as dimethylsulfoxide (DMSO), N,N-dimethylformamide (DMF), N,N-dimethylacetamide, tetrahydrofuran (THF), acetone, acetonitrile, or the like; alcohol solvents like methanol, ethanol, ethylene glycol, isopropyl alcohol, 2-methoxy ethanol or the like; or mixtures thereof. Suitable temperatures for the reactions of a) to e) range from about 0° C. to about the reflux temperature of the solvent, or in the range of about 25° C. to about 150° C.

The products obtained in each of the steps a) to e) may be isolated by techniques well known in the art, or they may be used for the subsequent reaction steps without isolation. The products may be isolated by adding water and separating the organic layer and aqueous layer, wherein the product is further isolated by evaporating the organic layer under vacuum, or the product may be isolated by direct filtration if the product is precipitated in the reaction mass. The isolated intermediates may be further purified by precipitation from a solvent or a mixture of solvents.

Suitable solvents which can be used for extraction of the product or precipitation include but are not limited to alcohols such as methanol, ethanol, 2-propanol, 1-butanol, tertiary-butanol, or the like; ethers such as diethyl ether, dimethyl ether, diisopropyl ether, tetrahydrofuran, 1,4 dioxane, or the like; aromatic hydrocarbons such as toluene, xylene, or the like; halogenated hydrocarbons such as dichloromethane, chloroform, carbon tetrachloride, chlorobenzene, or the like; ketones such as acetone, methyl isobutyl ketone, or the like; and any mixtures of such solvents and combinations with water, in various proportions.

Suitably, the products at each stage when isolated as solid can be dried. Drying can be suitably carried out in a tray dryer, vacuum oven, air oven, fluidized bed dryer, spin flash dryer, flash dryer, spray dryer, thin film dryer, or the like. The drying can be carried out at temperatures about 35° C. to about 90° C., or other temperatures suitable for removing the solvent without affecting purity of the product. The drying can be carried out for any desired time periods such as about 1 to 20 hours, or longer.

Fosamprenavir obtained can be suitably converted to any of its pharmaceutically acceptable salts, by reacting with a suitable reagent in the presence of a suitable solvent.

Suitable salts of fosamprenavir include, but are not limited to, calcium, disodium, dipotassium, magnesium, piperazine, etc.

In an embodiment, the salt is fosamprenavir calcium and it is prepared by reacting fosamprenavir with a calcium compound such as calcium acetate.

The calcium salt of fosamprenavir may be prepared by interconversion of salts, wherein a more basic salt may be reacted with a less basic fosamprenavir salt, to replace the weak base.

In an embodiment, a piperazine salt of fosamprenavir is converted to fosamprenavir calcium by reacting with calcium acetate.

The fosamprenavir salt can be obtained by reacting fosamprenavir with the salt forming compound, or it can be obtained directly from a reaction in which a fosamprenavir salt is formed.

A fosamprenavir salt obtained in the present application may be further purified by recrystallization or slurrying in a suitable solvent. Recrystallization involves providing a solution of a crude compound in a suitable solvent and then crystallizing the solid from the solution. Suitable solvents which can be used for purification include, but are not limited to: ketones such as acetone, ethyl methyl ketone, butanone, methyl isobutyl ketone or the like; alcohols such as methanol, ethanol, 2-butanol, tertiary-butanol, and 2-propanol; ethers such as tetrahydrofuran and 1,4-dioxane, esters such as ethyl acetate or the like; water; and any mixtures thereof.

The present application also provides intermediate compounds for the preparation of fosamprenavir.

In an embodiment the intermediate compound is N-((2R,3S)-3-amino-2-hydroxy-4-phenylbutyl)-4-(1,3-dioxoisoindolin-2-yl)-N-isobutylbenzenesulphonamide of Formula V, and salts thereof.

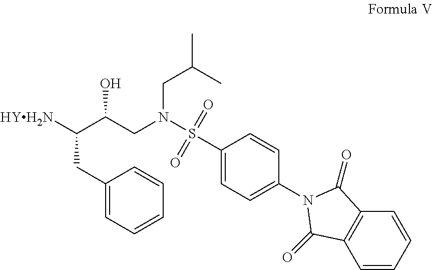

In another embodiment, the intermediate compound is (S)-tetrahydrofuran-3-yl-(2S,3R)-4-(4-(1,3-dioxoisoindolin-2-yl)-N-isobutylphenylsulphonamido)-1-phenyl-3-(phosphonooxy)-butan-2-ylcarbamate of Formula VII, and its salts.

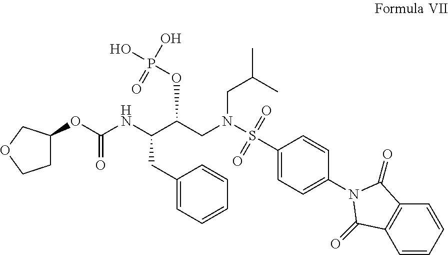

Another aspect of the present application provides a process for the preparation of fosamprenavir or its pharmaceutically acceptable salts comprising:
a) converting the phthalimide protected fosamprenavir of Formula VII to a pharmaceutically acceptable salt of Formula VIIIa:

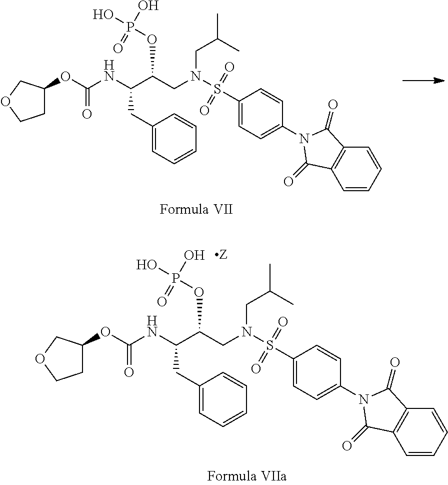

wherein Z is any pharmaceutically acceptable salt forming residue;
b) deprotecting the salt of Formula VIIIa to obtain a fosamprenavir salt; and
c) optionally, converting the fosamprenavir salt to fosamprenavir calcium, when the salt of b) is not calcium.

The salt forming residue Z in Formula VIIIa may be any of pharmaceutically acceptable salt-formers, for example, calcium, disodium, dipotassium, magnesium, zinc, ethylenediamine, piperazine, etc. without limitation.

Suitable solvents that can be used for the above reactions a) through c) include, but are not limited to: ether solvents such as tetrahydrofuran, 1,4-dioxane, or the like; halogenated hydrocarbon solvents such as dichloromethane, ethylene dichloride, or the like; alcohol solvents such as methanol, ethanol, 2-propanol, or the like; ketone solvents such as acetone, methyl isobutyl ketone, or the like; hydrocarbon solvents such as toluene, xylene, n-hexane, n-heptane, cyclohexane, or the like; ester solvents such as ethyl acetate, n-propyl acetate, isopropyl acetate, n-butyl acetate, tertiary-butyl acetate, or the like; aprotic polar solvents such as dimethylformamide, dimethylsulfoxide, dimethylacetamide, or the like; and any mixtures thereof or their combinations with water in various proportions, without limitation. Suitable temperatures for the reactions of a) to c) range from about 0° C. to about the reflux temperature of the solvent, or in the range of about 25° to about 150° C.

Another aspect of the present application provides a process for the preparation of substantially amorphous fosamprenavir calcium comprising recrystallizing from a solution of fosamprenavir calcium in an alcohol solvent.

The solution of fosamprenavir calcium may be obtained by dissolving any hydrate, solvate or any crystalline form of fosamprenavir calcium in a suitable alcohol solvent, or such a solution may be obtained directly from a reaction in which fosamprenavir calcium is formed.

Suitable alcohol solvents which can be used include, but are not limited to, methanol, ethanol, 2-propanol, 1-butanol, 1-propanol, tertiary-butyl alcohol, or the like, and any mixtures thereof.

Fosamprenavir and its salts when prepared according to a process of the present application may have a chemical purity greater than about 95% by weight, as determined using high performance liquid chromatography (“HPLC”).

The present application also provides pharmaceutical compositions comprising fosamprenavir and its salts prepared according to the processes of the present application, either alone or in combination with one or more pharmaceutically acceptable carrier, and further in combination with one or more pharmaceutically acceptable excipients.

The pharmaceutical composition comprising fosamprenavir or its salts and its combination with a pharmaceutically acceptable carrier of this application may further formulated as solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation, or wet granulation or by extrusion and spheronization. Compositions may be presented as uncoated, film coated, sugar coated, powder coated, enteric coated or modified release coated. Compositions of the present application may further comprise one or more pharmaceutically acceptable excipients.

Pharmaceutically acceptable excipients that find use in the present application include, but are not limited to: diluents such as starch, pregelatinized starch, lactose, powdered cellulose, microcrystalline cellulose, dicalcium phosphate, tricalcium phosphate, mannitol, sorbitol, sugar, or the like; binders such as acacia, guar gum, tragacanth, gelatin, polyvinyl pyrrolidone, hydroxypropyl cellulose, hydroxypropyl methylcellulose, pregelatinized starch, or the like; disintegrants such as starch, sodium starch glycolate, pregelatinized starch, crospovidone, croscarmellose sodium, colloidal silicon dioxide, or the like; lubricants such as stearic acid, magnesium stearate, zinc stearate, or the like; glidants such as colloidal silicon dioxide, or the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes, or the like. Other pharmaceutically acceptable excipients that are of use include but are not limited to film formers, plasticizers, colorants, flavoring agents, sweeteners, viscosity enhancers, preservatives, antioxidants, or the like.

In the compositions of present application fosamprenavir or its salts is a useful active ingredient in the range of 50 mg to 700 mg, per dosage unit

Fosamprenavir and its salts as described herein may have residual solvent contents in amounts that are within the limits given by the International Conference on Harmonization of Technical Requirements for Registration of Pharmaceuticals for Human Use (“ICH”) guidelines.

### DEFINITIONS

The following definitions are used in connection with the present invention unless the context indicates otherwise. “Amorphous” forms consist of disordered arrangements of molecules that do not possess a distinguishable crystal lattice. “Amine-protecting group” refers to a radical when attached to a nitrogen atom in a target molecule is capable of surviving subsequent chemical reactions applied to the target molecule i.e. hydrogenation, reaction with acylating agents, alkylation etc. The amine-protecting group can later be removed. Amine-protecting groups include, but are not limited to, fluorenylmethoxycarbonyl (FMOC), tert-butoxycarbonyl (t-BOC), benzyloxycarbonyl (Z), those of the acyl type (e.g., formyl, benzoyl, trifluoroacetyl, p-tosyl, aryl- and alkylphosphoryl, phenyl- and benzylsulfonyl, o-nitrophenylsulfenyl, o-nitrophenoxyacetyl), and of the urethane type (e.g. tosyloxyalkyloxy-, cyclopentyloxy-, cyclohexyloxy-, 1,1-dimethylpropyloxy, 2-(p-biphenyl)-2-propyloxy- and benzylthiocarbonyl). Amine-protecting groups are made using a reactive agent capable of transferring an amine-protecting group to a nitrogen atom in the target molecule. Examples of an amine-protecting agent include, but are not limited to, C₁-C₆ aliphatic acid chlorides or anhydrides, C₆-C₁₄arylcarboxylic acid chlorides or anhydrides, t-butyl chloroformate, di-tert-butyl dicarbonate, butoxycarbonyloxyimino-2-phenylacetonitrile, t-butoxycarbonyl azide, t-butyl fluoroformate, fluorenylmethoxycarbonyl chloride, fluorenylmethoxycarbonyl azide, fluorenylmethoxycarbonyl benzotriazol-1-yl, (9-fluorenylmethoxycarbonyl)succinimidyl carbonate, fluorenylmethoxycarbonyl pentafluorophexoxide, trichloroacetyl chloride, methyl-, ethyl-, trichloromethyl-chloroformate, and other amine-protecting agents known in the art. Examples of such known amino-protecting agents are found in pages 385-397 of T. W. Green and P. G. M. Wuts, “Protective Groups in Organic Synthesis, Second Edition”, Wiley-Interscience, New York, 1991.

An “alcohol solvent” is an organic solvent containing a carbon bound to a hydroxyl group. “Alcohol solvents” include but are not limited to methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, C₁₋₆alcohols, or the like.

An “amide solvent” is an organic solvent containing a carbonyl group (C═O) or phosphorus atom bonded to a nitrogen atom. “Amide solvents” include, but are not limited to, N,N-dimethylformamide (DMF), N,N-dimethylacetamide (DMA), N-methylpyrrolidone (NMP), formamide, acetamide, propanamide, 1,1,3,3-tetramethyl urea, N,N′-dimethylpropylene urea, hexamethyl phosphoramide (HMPA), or hexamethyl phosphorus triamide (HMPT).

An “aprotic polar solvent” has a dielectric constant greater than 15 and is at least one chosen from amide-based organic solvents, such as N,N-dimethylformamide (DMF), N,N-dimethylacetamide (DMA), N-methylpyrrolidone (NMP), formamide, acetamide, propanamide, hexamethyl phosphoramide (HMPA), or hexamethyl phosphorus triamide (HMPT); nitro-based organic solvents, such as nitromethane, nitroethane, nitropropane, or nitrobenzene; pyridine-based organic solvents, such as pyridine or picoline; sulfone-based solvents, such as dimethylsulfone, diethylsulfone, diisopropylsulfone, 2-methylsulfolane, 3-methylsulfolane, 2,4-dimethylsulfolane, 3,4-dimethy sulfolane, 3-sulfolene, or sulfolane; sulfoxide-based solvents such as dimethylsulfoxide (DMSO); ester-based organic solvents, such as γ-butyrolactone, ethylene carbonate, propylene carbonate, butylene carbonate, dimethyl carbonate, and propiolactone; or nitrile-based organic solvents, such as acetonitrile, propionitrile, or benzonitrile. These organic solvents may be used alone or two or more of these may be combined appropriately.

An “ester solvent” is an organic solvent containing a carboxyl group —(C═O)—O-bonded to two other carbon atoms. “Ester solvents” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, C₃₋₆esters, or the like.

An “ether solvent” is an organic solvent containing an oxygen atom —O— bonded to two other carbon atoms. “Ether solvents” include but are not limited to diethyl ether, diisopropyl ether, methyl t-butyl ether, glyme, diglyme, tetrahydrofuran, 1,4-dioxane, dibutyl ether, dimethylfuran, 2-methoxyethanol, 2-ethoxyethanol, anisole, C₂₋₆ethers, or the like.

A “halogenated solvent” is an organic solvent containing a carbon bound to a halogen. “Halogenated hydrocarbon solvents” include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

A “ketone solvent” is an organic solvent containing a carbonyl group —(C═O)-bonded to two other carbon atoms. “Ketone solvents” include, but are not limited to, acetone, ethyl methyl ketone, diethyl ketone, methyl isobutyl ketone, C₃₋₆ketones, or the like.

“Protecting group” means a chemical group that (a) preserves a reactive group from participating in an undesirable chemical reaction, and (b) can be removed after protection of the reactive group is no longer required.

“Carbonylation agent” refers to a compound, molecule, or substance, capable of reacting with two nucleophiles, which may be the same or different, to introduce carbonyl diradical into the product molecule along with radicals derived from the two nucleophiles. In some embodiments, the attacking nucleophiles are an amine or alcohol, resulting in carbamate, urea, or carbonate formation. Non-limiting examples of such carbonylation agents include 1,1′-carbonyldiimidazole; 1,1′-carbonyldi-1,2,3-benzotriazole; 1,1′-carbonyldi-1,2,4-triazole; C₁-C₆alkyl chloroformate reagents such as methyl chloroformate, ethyl chloroformate, propyl chloroformate, isopropyl chloroformate, or butyl chloroformate; allyl chloroformate; optionally substituted C₆-C₁₄aryl chloroformate reagents such as phenyl chloroformate, nitrophenyl chloroformate, or benzyl chloroformate; C₁-C₆alkyl carbonates such as dimethyl carbonate, diethyl carbonate, or di(tert-butyl) carbonate; carbamic acid halides such as N,N-dimethyl carbamic acid chloride; p-nitrophenyl carbamate; 2,4-dinitrophenyl carbonate; carbonylbis(methyl ethyl ketoxime); N,N′-disuccinimidyl carbonate; phosgene; diphosgene; and triphosgene.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), triethiodide, tromethamine (2-amino-2-(hydroxymethyl)-1,3-propanediol), valerate, and zinc salts.

“Phosphorylating agents” are compounds capable of transferring the group (HO)₂P(O) in free or esterified form. Non-limiting examples of such phosphorylating agents include phosphorus oxychloride, phosphorus pentachloride, phosphoric anhydride, phospho-tris-triazolides, phospho-tris-triimidazolides, dibenzylphosphite/carbon tetrabromide, bis(2,2,2-trichloroethyl) phosphorodichloridate, benzyl methyl phosphorochloridate, diphenylphosphoryl chloride, β-cyanoethyl phosphate, and dibenzylchlorophosphate.

Certain specific aspects and embodiments of the present application will be explained in more detail with reference to the following examples, which are provided only for purposes of illustration and should not be construed as limiting the scope of the application in any manner. Reasonable variations of the described procedures are intended to be within the scope of the present application.

### EXAMPLES

All XRPD data reported herein were obtained using a Bruker Axs D8 Advance Powder X-ray Diffractometer, with copper Kα radiation. Differential Scanning calorimetric analysis of substantially amorphous fosamprenavir calcium was carried out in a DSC Q1000 model from TA Instruments.

### Example 1

Preparation of tert-butyl-(2S,3R)-3-hydroxy-4-(isobutylamino)-1-phenylbutan-2-yl carbamate (Formula II). Tert-butyl-(S)-1-((S)-oxiran-2-yl)-2-phenylethylcarbamate (200 g) was placed into a round bottom flask and methanol (1000 mL) was added, followed by addition of isobutylamine (284.7 g). The mixture was heated to reflux and maintained for about 3 hours. Reaction completion was determined using thin layer chromatography. After the reaction completed, about 80% of the methanol was distilled and water (2000 mL) was added to the residue. The mixture was then cooled to about 25° C. The formed solid was filtered and washed with water (400 mL). The wet solid was dried at about 65° C. for 12-15 hours until the moisture content was less than 1%, to yield 246.4 g of the title compound. Purity by HPLC: 95.54%.

### Example 2

Preparation of N-((2R,3S)-3-amino-2-hydroxy-4-phenylbutyl)-4-(1,3-dioxoisoindolin-2-yl)-N-isobutylbenzenesulfonamide hydrochloride (Formula V). Tert-butyl-(2S,3R)-3-hydroxy-4-(isobutylamino)-1-phenylbutan-2-yl carbamate (50 g) was heated with toluene (350 mL) at 80-85° C., and triethylamine (16.6 mL) was added. The mixture was maintained at 80-85° C. and a suspension of 4-(1,3-dioxoisoindolin-2-yl)-benzene-1-sulfonyl chloride (40.5 g) in toluene (100 mL) was added. The mixture was stirred at 85-90° C. until reaction completion. Concentrated HCl (12.5 mL) was added by drops, the mass was maintained at 85-90° C. for 1 hour, a further quantity of concentrated HCl (10 mL) was added by drops, and the mass was maintained at the same temperature for about 30 minutes. The mass was cooled to 60-70° C. and the solvent was completely distilled under vacuum. Ethanol (350 mL) was added to the residue and stirred for 30 minutes. The solution was cooled slowly to 0-5° C. and the formed solid was isolated by filtration, washed with ethanol (75 mL), and dried at about 60° C. under vacuum for 7 hours to produce 54.6 g of the product. Purity by HPLC: 98.48%; m/z: 522.1 [M+H⁺].

### Example 3

Preparation of N-((2R,3S)-3-amino-2-hydroxy-4-phenylbutyl)-4-(1,3-dioxoisoindolin-2-yl)-N-isobutylbenzenesulfonamide hydrochloride (Formula V). Tert-butyl-(2S,3R)-3-hydroxy-4-(isobutylamino)-1-phenylbutan-2-yl carbamate (100 g) was mixed with dichloromethane (500 mL) at 20-35° C., triethylamine (41 mL) was added, then dimethylaminopyridine (0.2 g) and 4-(1,3-dioxoisoindolin-2-yl)-benzene-1-sulfonyl chloride (95.33 g) were added. The mixture was stirred at 25-35° C. until reaction completion. After reaction completion, the solvent was completely distilled at 40° C. under vacuum, to produce a thick residue. To the residue, isopropyl alcohol (100 mL) was added and distilled to remove residual dichloromethane. To the residue, isopropyl alcohol (1500 mL) was added, followed by drop-wise addition of concentrated HCl (75 mL). The mass was heated to 75-80° C. and maintained for 2-3 hours. After reaction completion, the mass was cooled to about 20° C. and the formed solid was filtered and washed with isopropyl alcohol (200 mL). The wet solid was dried at about 50° C. for about 5 hours, to yield 154.6 g of the title compound. Purity by HPLC: 99.8%; m/z: 522.1 [M+H⁺].

### Example 4

Preparation of (S)-tetrahydrofuran-3-yl(2S,3R)-4-(4-(1,3-dioxoisoindolin-2-yl)-N-isobutylphenylsulfonamido)-3-hydroxy-1-phenylbutan-2-yl carbamate (Formula VI). 1,1′-Carbonyldiimidazole (13.6 g) was added to ethyl acetate (200 mL), followed by the addition of (S)-3-hydroxytetrahydrofuran (33.8 g). The mixture was maintained for 60-90 minutes at ambient temperature. N-((2R,3S)-3-amino-2-hydroxy-4-phenylbutyl)-4-(1,3-dioxoisoindolin-2-yl)-N-isobutylbenzenesulfonamide hydrochloride (40 g) was added and the mixture heated to reflux for about 15-20 hours. The mass was cooled to ambient temperature and water (240 mL) was added and stirred. The layers were separated and the organic layer was washed with 50% aqueous HCl solution (120 mL) and water (120 mL). The organic layer was dried with anhydrous sodium sulfate and was completely distilled under vacuum. Ethanol (120 mL) was added to the residue and cooled to 20° C. The solid was filtered and washed with ethanol (40 mL). The wet solid was mixed with ethanol (120 mL) and water (20 mL) and stirred for 15 minutes. The solid was isolated by filtration, washed with petroleum ether (100 mL), and dried at about 50° C. under vacuum for 1 hour, to yield 14.6 g of product. Purity by HPLC: 95.54%.

### Example 5

Preparation of (S)-tetrahydrofuran-3-yl(2S,3R)-4-(4-(1,3-dioxoisoindolin-2-yl)-N-isobutylphenylsulfonamido)-3-hydroxy-1-phenylbutan-2-ylcarbamate (Formula VI). To a solution of 10.86 g of carbamate, prepared by reacting (S)-tetrahydro-3-furanol with 4-nitrophenylchloroformate in dichloromethane (200 mL), pyridine (20 mL) and diisopropylethylamine (12.44 mL) were added, followed by the addition of N-((2R,3S)-3-amino-2-hydroxy-4-phenylbutyl)-4-(1,3-dioxoisoindolin-2-yl)-N-isobutylbenzenesulfonamide hydrochloride (20 g) and 1-Hydroxybenzotriazole (2.35 g). The mixture was maintained at ambient temperature until reaction completion, and then was quenched by adding a solution of 5% dilute hydrochloric acid (1000 mL). The layers were separated and the organic layer was washed with water (2×500 mL), 20% sodium bicarbonate solution (5×500 mL), and finally with water (3×500 mL). The organic layer was separated, dried with anhydrous sodium sulfate, and distilled under vacuum. The residue was slurried in isopropyl alcohol (110 mL) and ethyl acetate (40 mL); stirred for about two hours, filtered, and the solid washed with isopropyl alcohol (40 mL). The wet solid was dried under vacuum at about 50° C., to yield 11.5 g of the title compound. Purity by HPLC: 97.57%.

### Example 6

Preparation of (S)-tetrahydrofuran-3-yl-(2S,3R)-4-(4-(1,3-dioxoisoindolin-2-yl)-N-isobutylphenylsulfonamido)-1-phenyl-3-(phosphonooxy)-butan-2-ylcarbamate (Formula VII). Pyridine (80 mL) and (S)-tetrahydrofuran-3-yl-(2S,3R)-4-(4-(1,3-dioxoisoindolin-2-yl)-N-isobutylphenylsulfonamido)-3-hydroxy-1-phenylbutan-2-ylcarbamate (8 g) were stirred and heated to a temperature of 50° C. The solvent was distilled to about 50% of its original volume. The mass was cooled to about 0-5° C. and POCl₃ (4.8 mL) was added slowly, then the mass was maintained at ambient temperature for about 2-3 hours. The mass was cooled to about 0-5° C., water (80 mL) and methyl isobutyl ketone (50 mL) were added, the mixture was stirred, and the layers were separated. The aqueous layer was extracted with methyl isobutyl ketone (50 mL). The combined organic layer was washed with 5% aqueous HCl solution (20 mL). The aqueous layer was again with methyl isobutyl ketone (40 mL). The organic extracts were combined and reacted with 5% aqueous HCl solution (50 mL) at 45-50° C. until reaction completion. The organic and aqueous layers were separated and the organic layer was washed with brine solution, and evaporated under vacuum to obtain 7.3 g of the product. Purity by HPLC: 92.54%; m/z: 715.2 [M+W].

### Example 7

Preparation of fosamprenavir. Methylamine in methanol (10%, 3.8 mL) was added to a solution of (S)-tetrahydrofuran-3-yl-(2S,3R)-4-(4-(1,3-dioxoisoindolin-2-yl)-N-isobutylphenyl-sulfonamido)-1-phenyl-3-(phosphonooxy)-butan-2-ylcarbamate (7.0 g) in methanol (70 mL). The mixture was maintained at 45-50° C. for about 60-90 minutes, then cooled to 35-40° C. The solvent was distilled under vacuum, methyl isobutyl ketone (105 mL) and water (150 mL) were added, and then sodium bicarbonate solution (2 g in 15 mL water) was added. The mass was stirred and the layers separated. The aqueous layer was washed with ethyl acetate (2×50 mL) and the pH was adjusted to 1.2-1.3 by adding aqueous HCl (5 mL). The aqueous layer was extracted with methyl isobutyl ketone (200 mL) and the solvent was distilled under vacuum, to yield 4.1 g of the product. Purity by HPLC: 87.36%.

### Example 8

Preparation of fosamprenavir calcium. A solution of fosamprenavir (10 g) in ethanol (50 mL) was heated to reflux and a solution of piperazine (2.94 g) in ethanol (40 mL) was added slowly under reflux conditions. The mass was maintained under reflux for about 45 minutes and then cooled to ambient temperature. Ethyl acetate (1000 mL) was added and the solvent mixture was completely distilled under vacuum. Ethyl acetate (100 mL) was added to the residue and the solid was isolated by filtration, washed with ethyl acetate (50 mL), and dried. The obtained piperazine salt of fosamprenavir was dissolved in ethanol (48 mL) and a solution of calcium acetate in water (1.88 g in 48 mL water) was added at about 50° C. The mixture was cooled to about 20-25° C. and the solid was isolated by filtration, washed with a mixture of ethanol (15 mL) and water (15 mL), and then with water (15 mL). The solid was further dried under vacuum at about 30° C. for about 90 minutes, to yield 5.0 g of the title compound. Purity by HPLC: 94.35%.

### Example 9

Preparation of phthalimide-protected fosamprenavir calcium. Ethanol (240 mL) and phthalimide-protected fosamprenavir (24 g) were placed into a round bottom flask and heated to about 50° C. Calcium acetate (5.3 g) was dissolved in water (240 mL) and slowly added at the same temperature. The mass was then cooled to 25-30° C. and maintained for about 5 hours. The formed solid was filtered and washed, first with a 1:1 mixture ethanol and water (48 mL) and finally with water (24 mL). The wet solid was dried at about 50° C. to a constant weight, to yield 13.2 g of the title compound. Purity by HPLC: 86.9%.

### Example 10

Preparation of fosamprenavir calcium. Methanol (66 mL) and phthalimide-protected fosamprenavir calcium (11 g) were placed into a round bottom flask and methylamine solution in methanol (10%, 4.5 mL) was added. The mass was heated to reflux, and maintained until reaction completion. After the reaction completed, the mass was cooled to about 35-40° C. and water (44 mL) was added, then the mass was cooled to about 25 to 35° C. and maintained for about 40 minutes. The formed solid was filtered and washed, first with a 1:1 mixture of methanol and water (22 mL), and finally with water (11 mL). The wet solid was dried at about 30° C. for about 2 to 3 hours, to yield 7.2 g of the title compound. 6.0 g of this compound was further recrystallized from a mixture of ethanol (84 mL) and water (6 mL) and washed with 1:1 ethanol and water (12 mL) to obtain 5.0 g of pure compound. Purity by HPLC: 97.88%.

### Example 11

Purification of fosamprenavir calcium. Fosamprenavir calcium (65 g) was placed into a round bottomed flask and ethanol (927 mL) and methanol (48 mL) were added. The mixture was heated to about 75° C. for about 20 minutes. The mixture was filtered and washed with a mixture of ethanol (309 mL) and methanol (16 mL). The filtrate was reheated to about 75° C. and water (195 mL) was added slowly in about 25 minutes. The mixture was cooled to about 25° C. and the formed solid was filtered and washed with a mixture of ethanol (130 mL) and water (130 mL). The wet compound was dried under vacuum to obtain 55 g of the required product. Purity by HPLC: 96.12%

### Example 12

Preparation of substantially amorphous fosamprenavir calcium. Fosamprenavir calcium from example 11 (3 g) was placed into a round bottom flask and ethanol (60 mL) was added. The mixture was heated to about 75° C. and stirred for about 30 minutes. The mixture was then cooled to about 25° C. and maintained for about 30 minutes. The formed solid was filtered and washed with ethanol (6 mL). The wet solid was dried at about 50° C. for about 2 hours to yield 1.3 g of the title compound. Purity by HPLC: 94.63%.

### Example 13

Preparation of substantially amorphous fosamprenavir calcium. Fosamprenavir calcium from example 10 (1.5 g) and methanol (50 mL) were placed into a round bottom flask and the solvent was distilled under vacuum at about 50° C. The distillation was repeated 5 times with the same quantities of methanol, to yield 1.1 g of the title compound. Purity by HPLC: 97.14%.

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound of Formula II: 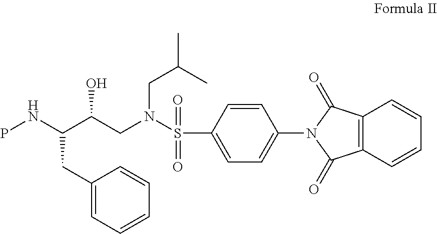
wherein P is an amine protecting group.

2. A process comprising reacting an amino alcohol of Formula III with 4-phthalimidobenzene sulfonyl halide of Formula IV: 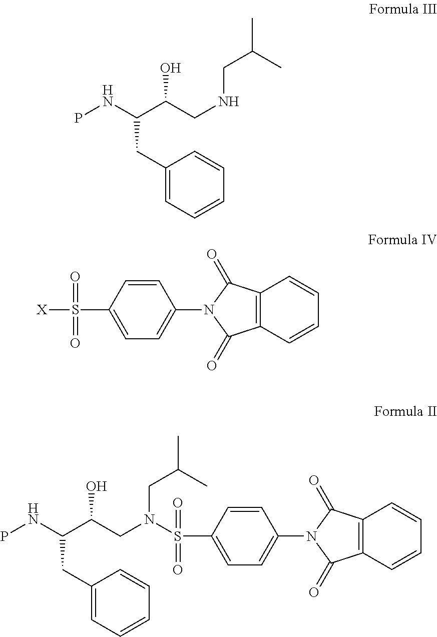
wherein X is Cl, Br, or I, and P is an amine protecting group;
to give the amine-protected N-((2R,3S)-3-amino-2-hydroxy-4-phenylbutyl)-4-(1,3-dioxoisoindolin-2-yl)-N-isobutylbenzenesulphonamide of Formula II.

3. A process comprising:
a) deprotecting the protected compound of Formula II in an acidic media to yield a compound of Formula V or a salt thereof: 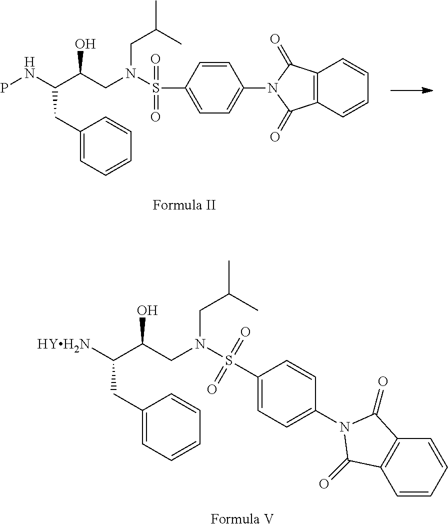
wherein Y is any acid addition salt-forming residue;
b) reacting the compound of Formula V with a derivative of (S)-tetrahydro-3-furanol or (S)-tetrahydro-3-furanol and optionally a carbonylation agent, to produce the phthalimide derivative of Formula VI; 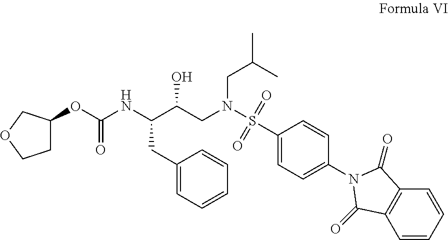
c) reacting the compound of Formula VI with a phosphorylating agent to obtain a phthalimide-protected fosamprenavir of Formula VII; 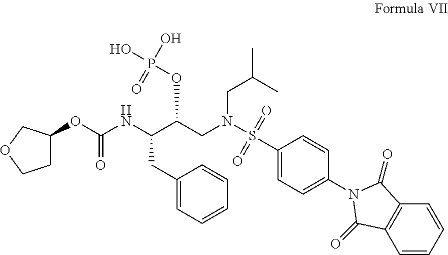
d) deprotecting the compound of Formula VII, to obtain fosamprenavir of Formula VIII; and 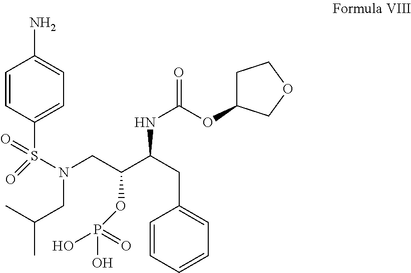
e) optionally, converting fosamprenavir to a pharmaceutically acceptable salt.

4. The process of claim 3, wherein the acidic media is step a) comprises hydrochloric acid.

5. The process of claim 3, wherein the reacting in step b) uses (S)-tetrahydro-3-furanol.

6. The process of claim 3, wherein the phosphorylating agent in step c) is phosphorus oxychloride.

7. The process of claim 3, wherein step d) is carried out using methylamine.

8. A process for the preparation of substantially amorphous fosamprenavir calcium comprising recrystallizing fosamprenavir calcium in an alcohol solvent.

9. The process of claim 8, wherein the alcoholic solvent is methanol, ethanol, 2-propanol, 1-butanol, 1-propanol, tertiary-butyl alcohol, or the like, or any mixtures thereof.

10. The process of claim 9, wherein the alcohol solvent is ethanol.

11. The compound N-((2R,3S)-3-amino-2-hydroxy-4-phenylbutyl)-4-(1,3-dioxoisoindolin-2-yl)-N-isobutylbenzenesulfonamide or its salts.

12. The compound (S)-tetrahydrofuran-3-yl-(2S,3R)-4-(4-(1,3-dioxoisoindolin-2-yl)-(S)-tetrahydrofuran-N-isobutylphenylsulphonamido)-1-phenyl-3-(phosphonooxy)-butan-2-ylcarbamate or its salts.
